(19) United States
(12) Patent Application Publication
(10) Pub. No.: US 2021/0022838 A1
(43) Pub. Date: Jan. 28, 2021

(54) METHOD OF CLEANING OR OTHERWISE TREATING TEETH

(71) Applicant: SUNSTAR AMERICAS, INC., Schaumburg, IL (US)

(72) Inventors: Kana Hashimoto, Mount Prospect, IL (US); Toru Saito, Buffalo Grove, IL (US)

(21) Appl. No.: 16/935,652

(22) Filed: Jul. 22, 2020

Related U.S. Application Data
(60) Provisional application No. 62/876,921, filed on Jul. 22, 2019.

(51) Int. Cl. A61C 17/20; A61C 17/02; A61Q 11/00

(52) U.S. Cl. CPC A61C 17/20; A61C 2201/00; A61Q 11/00; A61C 17/02

(57) ABSTRACT

A method of treating teeth and gums within an oral cavity including treating the accessible surfaces and hard-to-reach surfaces of the teeth and gums. The method includes providing an oral care composition including a therapeutic amount of sodium bicarbonate, providing a tool that generates hydrodynamic power, and applying the oral care composition to the accessible surfaces and the hard-to-reach surfaces of the teeth and gums and generating hydrodynamic power within the oral cavity. The combination of the oral care composition and the hydrodynamic power more effectively removes biofilm from the accessible surfaces and the hard-to-reach surfaces of the teeth and gums.

### CROSS-REFERENCE TO RELATED APPLICATIONS

This application claims the benefit of prior-filed, co-pending U.S. Provisional Patent Application No. 62/876,921, filed Jul. 22, 2019, the entire contents of which is incorporated by reference herein.

### FIELD OF INVENTION

The present invention relates to a method of cleaning or otherwise treating teeth.

### SUMMARY

One embodiment discloses a method of treating teeth and gums within an oral cavity including treating the accessible surfaces and hard-to-reach surfaces of the teeth and gums. The method includes providing an oral care composition including a therapeutic amount of sodium bicarbonate, providing a tool that generates hydrodynamic power, and applying the oral care composition to the accessible surfaces and the hard-to-reach surfaces of the teeth and gums and generating hydrodynamic power within the oral cavity. The combination of the oral care composition and the hydrodynamic power more effectively removes biofilm from the accessible surfaces and the hard-to-reach surfaces of the teeth and gums.

Another embodiment discloses a method of treating teeth and gums within an oral cavity including treating the accessible surfaces and hard-to-reach surfaces of the teeth and gums. The method includes providing an oral care composition including a therapeutic amount of sodium bicarbonate, providing a power toothbrush, and applying the oral care composition to the accessible surfaces and the hard-to-reach surfaces of the teeth and gums using the power toothbrush. The combination of the oral care composition and the power toothbrush more effectively removes biofilm from the accessible surfaces and the hard-to-reach surfaces of the teeth and gums.

Another embodiment discloses kit for treating teeth and gums within an oral cavity. The kit includes a tool for generating hydrodynamic power, and an oral care composition including a therapeutic amount of sodium bicarbonate. The oral care composition is adapted to be applied to the teeth and gums. The hydrodynamic power generated within the oral cavity by the tool in combination with the oral care composition removes biofilm from accessible surfaces and hard-to-reach surfaces of the teeth and gums. The sodium bicarbonate in the oral care composition, when combined with the hydrodynamic power, more effectively removes biofilm.

Another embodiment discloses a kit for treating teeth and gums within an oral cavity. The kit includes a power toothbrush and an oral care composition. The power toothbrush includes a handle, a plurality of bristles supported by the handle, and a power source supported by the handle. The power source is configured to drive movement of the plurality of bristles. The oral care composition includes a therapeutic amount of sodium bicarbonate and is adapted to be applied to the teeth by the power toothbrush. Using the power toothbrush in combination with the oral care composition removes biofilm from surfaces directly contacted by one or more of the plurality of bristles and generates hydrodynamic power that removes biofilm from hard-to-reach surfaces of the teeth and gums. The sodium bicarbonate in the oral care composition when combined with the hydrodynamic power more effectively removes biofilm.

Another embodiment discloses a combination for treating teeth and gums within an oral cavity. The combination includes a tool that generates hydrodynamic power, and an oral care composition including a therapeutic amount of sodium bicarbonate. The oral care composition is adapted to be applied to the teeth and gums. The hydrodynamic power generated by the tool within an oral cavity in combination with the oral care composition removes biofilm from accessible surfaces and hard-to-reach surfaces of the teeth and gums. The sodium bicarbonate in the oral care composition, when combined with the hydrodynamic power, more effectively removes biofilm.

Another embodiment discloses a combination for treating teeth and gums within an oral cavity including a power toothbrush and oral care composition including a therapeutic amount of sodium bicarbonate. The power toothbrush includes a handle, a plurality of bristles supported by the handle, and a power source supported by the handle. The power source is configured to drive the movement of the plurality of bristles. Using the power toothbrush in combination with the oral care composition removes biofilm from surfaces directly contacted by one or more of the plurality of bristles and generates hydrodynamic power that removes biofilm from hard-to-reach surfaces of the teeth and gums. The sodium bicarbonate in the oral care composition when combined with the hydrodynamic power more effectively removes biofilm.

Other aspects of the invention will become apparent by consideration of the detailed description and accompanying figures.

### BRIEF DESCRIPTIONS OF THE DRAWINGS

FIG. 1 illustrates the experimental setup of Experiment 1, discussed below.

FIG. 2 graphically shows the results of Experiment 1.

Before any embodiments of the invention are explained in detail, it is to be understood that the invention is not limited in its application to the details set forth in the following description. The invention is capable of other embodiments and of being practiced or of being carried out in various ways.

### DETAILED DESCRIPTION

Removing and preventing biofilm formation is the key to maintaining oral health. Biofilm can consist of multiple species of bacteria excreting a slimy glue-like substance that sticks to the surface of teeth. Some oral care compositions include antimicrobial agents, which kill planktonic bacteria and thereby help prevent biofilm formation. However, antimicrobial agents kill both commensal and pathogenic bacteria, and therefore may disrupt the oral ecosystem by allowing disease-promoting bacteria to manifest and cause oral issues. Although antimicrobial agents may help prevent biofilm formation, antimicrobial agents have a limited effect in removing existing biofilm because inhabitants of biofilm are effectively protected within this dense structure from host defense mechanisms. The most efficient measure to remove biofilm is mechanical scrubbing by a toothbrush. However, it is difficult to effectively mechanically scrub hard-to-reach areas, such as interproximal or gingival pocket spaces. Hydrodynamic power generated by a tool (e.g., a power toothbrush, an ultrasonic scaler, or a dental water irrigator, which is often referred to as a water flosser) can help remove some plaque at difficult to reach areas. However, hydrodynamic power does not remove as much plaque as physical scrubbing. Sodium bicarbonate is widely used in oral care as an anti-plaque (anti-biofilm) and anti-calculus component.

Biofilm may be more effectively removed from surfaces of the teeth and gums using a method in which a toothpaste composition including sodium bicarbonate is used in combination with a tool (e.g., a power toothbrush, an ultrasonic scaler, or a dental water irrigator) that generates hydrodynamic power. As used herein power toothbrush may include an electric toothbrush or a sonic toothbrush. An exemplary tool is shown as a power toothbrush 2 in FIG. 2. The power toothbrush 2 may include a handle 4, a plurality of bristles 6 supported by the handle 4, and a power source 8 supported by the handle 4. The power source 8 is configured to drive movement (e.g., rotational movement, pulsing movement, vibrational movement, sonic movement, ultrasonic movement, etc.) of the plurality of bristles 6. The method of treating teeth addresses biofilm removal from accessible surfaces of the teeth and gums and generally hard-to-reach surfaces of the teeth and gums. Accessible surfaces of the teeth and gums are those surfaces that are directly contacted by bristles 6 of the power toothbrush 2. Hard-to-reach surfaces of the teeth and gums are those that are partially contacted or not contacted by the bristles 6 of the power toothbrush 2. Exemplary hard-to-reach surfaces include, but are not limited to, interproximal surfaces of the teeth, surfaces in the gingival pockets, and lingual surfaces of the teeth. The method includes providing an oral care composition including a therapeutic amount of sodium bicarbonate and providing tool (e.g., the power toothbrush 2, an ultrasonic scaler, or a dental water irrigator) that generates hydrodynamic power. The method further includes applying the oral care composition to the accessible surfaces and the hard-to-reach surfaces of the teeth and gums, and generating hydrodynamic power within the oral cavity. The combination of the hydrodynamic power generated by the tool and the oral care composition removes biofilm from accessible surfaces and hard-to-reach surfaces of the teeth and gums. In particular, the sodium bicarbonate is present in the oral care composition in a concentration that enhances the hydrodynamic power to more effectively remove biofilm from the hard-to-reach surfaces, as will be discussed in greater detail below.

In some embodiments, applying the oral care composition to the accessible surfaces and the hard-to-reach surfaces of the teeth and gums is accomplished by using the power toothbrush 2. In the power toothbrush embodiment, the method includes mechanically scrubbing the accessible surfaces of the teeth and gums with the bristles of the power toothbrush to remove biofilm, and generating, by the power toothbrush, hydrodynamic power to remove biofilm from hard-to-reach surfaces of the teeth and gums. The combination of the oral care composition and the power toothbrush more effectively removes biofilm from the accessible surfaces and the hard-to-reach surfaces of the teeth and gums.

In some embodiments, the user may apply the oral care composition (as a toothpaste, a gel, a mouthwash, or other liquid form) to the accessible surfaces and the hard-to-reach surfaces of the teeth and gums prior to use of the tool. Then, the tool may be used to generate the hydrodynamic power. In the case of a dental water irrigator, for example, water may be expelled therefrom towards the accessible surfaces and hard-to-reach surfaces of the teeth and gums to generate the hydrodynamic power, which in combination with the oral care composition more effectively removes biofilm from the accessible surfaces and the hard-to-reach surfaces of the teeth and gums. In still other embodiments, the oral care solution may be expelled from the dental water irrigator in lieu of or in addition to water.

### Experiment 1

Experiment 1 evaluated non-contact biofilm removal by treating laboratory-created biofilms with different samples of the oral care compositions, as noted below and represented in FIG. 2, in combination with hydrodynamic power created by a power toothbrush.

The procedure for creating a biofilm used for Experiment 1 is as follows. That is, a Streptococcus mutans (“S mutans”) solution was cultured in a brain heart infusion (“BHI”) medium overnight at 37 degrees Celsius and under anaerobic conditions. Then, the S mutans solution was diluted with the BHI medium with 0.5% sucrose to adjust the number of bacteria to 10⁸ cfu/ml. This step ensures that the S mutans concentration is consistent for each sample. Then, the S mutans solution was inoculated on each of multiple hydroxyapatite discs 10, and cultured for 24 hours at 37 degrees Celsius under anaerobic conditions to form the biofilm 14. The biofilm 14 on each of the hydroxyapatite discs was washed with phosphate-buffered saline (“PBS”) once.

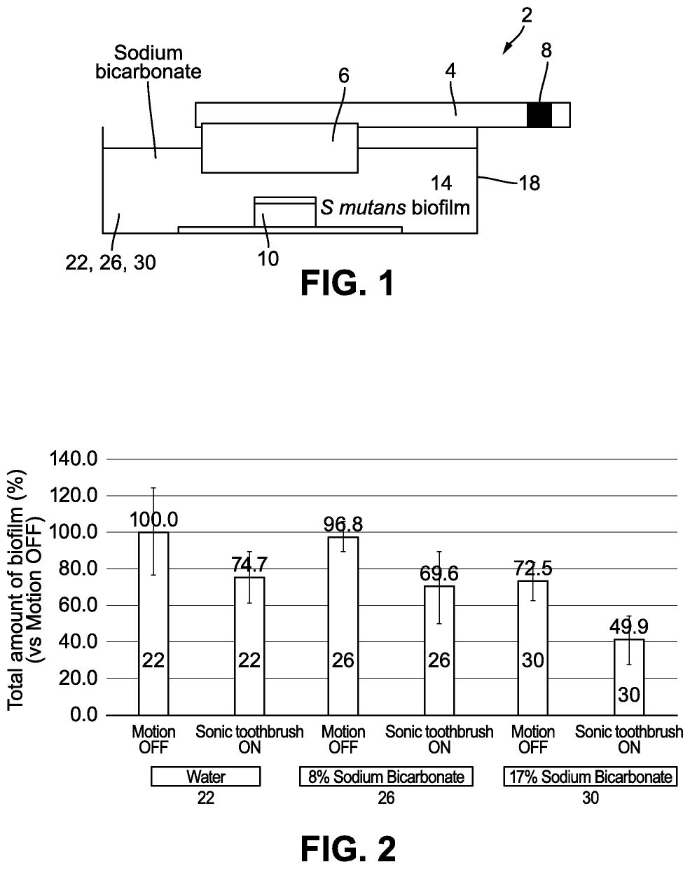

Then, as shown in FIG. 1, the biofilms 14, via the hydroxyapatite discs 10, were each placed in a culture dish 18 and each of the biofilms 14 were exposed to 40 ml of each of the samples (represented by reference numerals 22, 26, 30 in FIG. 1) such that each of the samples covered and surrounded the lab-created biofilm 14 and the hydroxyapatite disc 10. As shown in FIG. 2, Sample 1 (represented by reference numeral 22) included water alone, Sample 2 (represented by reference numeral 26) included about 8% sodium bicarbonate by weight, and Sample 3 (represented by reference numeral 30) included about 17% sodium bicarbonate by weight. Two sets of tests were run. The first test merely exposed the biofilms 14 to each of the samples for a period of 30 seconds. The second test exposed the biofilm to each of the samples and applied hydrodynamic power via a power toothbrush 2 to the samples for 30 seconds. The power toothbrush 2 used in Experiment 1 was a sonic toothbrush. Accordingly, as shown in FIG. 1, the power toothbrush 2, which was spaced apart from the lab-created biofilm 14 by about 2 mm, applied hydrodynamic power to each of the samples 22, 26, 28 for 30 seconds. See the American Journal of Dentistry, Vol. 10, pp. 65-69, 2010. Then, for each set of tests, the exposed-biofilms 14 were each washed with PBS once. Finally, the samples were removed from each culture dish 18 and each of the exposed-biofilms 14 were dissolved with 200 μL 1N NaOH. Then the efficacy of each solution to remove the biofilm 14 on the respective hydroxyapatite disc 10 was measured using UV-vis spectrophotometric optical density at a wavelength of 550 nm (OD550).

As shown in FIG. 2, Sample 1, containing only water, least effectively removed biofilm. That is, about 100% biofilm remained after being treated with water alone. Moreover, Sample 1 in combination with movement of the power toothbrush removed slightly more biofilm. That is, about 74.7% biofilm remained after being treated with water and a power toothbrush. On the other hand, Sample 3, containing 17% sodium bicarbonate, in combination with the movement of a power toothbrush removed the most biofilm. That is, about 40.9% biofilm remained after being treated with Sample 3 including 17% bicarbonate and a power toothbrush. Sample 2, containing 8% sodium bicarbonate, in combination with the movement of a power toothbrush was also effective at removing biofilm, though not as effective as Sample 3. That is, about 69.6% biofilm remained after being treated with Sample 2 including 8% sodium bicarbonate and a power toothbrush. Notably, the results appear to indicate the combination of sodium bicarbonate and hydrodynamic power of a power toothbrush are more effective at removing biofilm that merely using sodium bicarbonate alone. This combination may allow plaque control technology to shift from destruction of biofilm without killing bacteria using antimicrobial agents.

Experiment 1 was carried out using an aqueous solution having the concentrations reported above. In actual oral care products using sodium bicarbonate, the concentrations may vary according to the specific oral care product. For example, when the oral care composition is a toothpaste, the concentrations of sodium bicarbonate may be increased because the toothpaste when used by a user will be diluted by saliva to about as much as ½ to ⅕. In one specific embodiment, when used in a toothpaste, for example, the concentration of sodium bicarbonate may be 51% by weight, which is determined by the amount of dilution that typically may occur during normal use by a user. When toothpaste having 51% by weight sodium bicarbonate is diluted by saliva it generally may result in a solution having about 17% sodium bicarbonate with an estimated ⅓ dilution rate. In some embodiments, when used in a toothpaste, for example, the concentration of sodium bicarbonate may range from 24% by weight to 51% by weight, which is determined by the amount of dilution that typically may occur during normal use by a user. When toothpaste having 24% by weight sodium bicarbonate is diluted by saliva it generally may result in a solution having about 8% sodium bicarbonate with an estimated ⅓ dilution rate. Likewise, in some embodiments, when used in a toothpaste, for example, the concentration of sodium bicarbonate may range from 16% by weight to 85% by weight, which is determined by the amount of dilution that typically may occur during normal use by a user. When toothpaste having 16% by weight sodium bicarbonate is diluted by saliva it generally may result in a solution having about 8% sodium bicarbonate with an estimated ½ dilution rate, and when toothpaste having 85% by weight sodium bicarbonate is diluted by saliva it generally may result in a solution having about 17% sodium bicarbonate with an estimated ⅕ dilution rate. In other embodiments, sodium bicarbonate may be present in a suitable concentration such that when used in a toothpaste and combined with saliva the resulting solution has sodium bicarbonate ranging from 8% by weight to 17% by weight. Alternatively, when the oral care composition is a gel, mouthwash, or other liquid form, it may not be diluted as much during use as a toothpaste so the concentration of sodium bicarbonate may remain closer to the concentrations used in Experiment 1. Accordingly, in some embodiments, when used as a gel, mouthwash, or other liquid form, the concentration of sodium bicarbonate may be 17% by weight. In other embodiments, when used in a gel, mouthwash, or other liquid form, the concentration of sodium bicarbonate may range from 8% by weight to 17% by weight. In other embodiments, sodium bicarbonate may be present in a suitable concentration such that when used in a gel, a mouthwash, or other liquid form and combined with saliva the resulting solution has sodium bicarbonate ranging from 8% by weight to 17% by weight.

Although various aspects have been described in detail with reference to certain embodiments, variations and modifications exist within the scope and spirit of one or more independent aspects as described. Various features and advantages are set forth in the following claims.

1. A method of treating teeth and gums within an oral cavity including treating the accessible surfaces and hard-to-reach surfaces of the teeth and gums, the method comprising:
providing an oral care composition including a therapeutic amount of sodium bicarbonate;
providing a tool that generates hydrodynamic power; and
applying the oral care composition to the accessible surfaces and the hard-to-reach surfaces of the teeth and gums and generating hydrodynamic power within the oral cavity,
wherein the combination of the oral care composition and the hydrodynamic power more effectively removes biofilm from the accessible surfaces and the hard-to-reach surfaces of the teeth and gums.

2. The method of claim 1, wherein the tool is a power toothbrush, an ultrasonic scaler, or a dental water irrigator.

3. The method of claim 1, wherein the tool is a power toothbrush and further comprising
mechanically scrubbing the accessible surfaces of the teeth and gums with the bristles of the power toothbrush to remove biofilm, and
generating, by the power toothbrush, the hydrodynamic power to remove biofilm from hard-to-reach surfaces of the teeth and gums,
wherein the sodium bicarbonate in the oral care composition when combined with the hydrodynamic power more effectively removes biofilm.

4. The method of claim 1, wherein the hard-to-reach surfaces of the teeth include interproximal surfaces of the teeth, surfaces in the gingival pockets, and lingual surfaces of the teeth.

5. The method of claim 1, wherein the oral care composition is a toothpaste and a concentration of sodium bicarbonate in the oral care composition is between 24% and 51% by weight.

6. The method of claim 1, wherein the oral care composition is a gel, mouthwash, or other liquid form and a concentration of sodium bicarbonate in the oral care composition is between 8% and 17% by weight.

7. The method of claim 1, wherein a concentration of sodium bicarbonate in the oral care composition is between 8.0% by weight and 51% by weight.

8. The method of claim 1, wherein the oral care composition is free of antimicrobial agents.

9. A kit for treating teeth and gums within an oral cavity, the kit comprising:
a tool for generating hydrodynamic power; and
an oral care composition including a therapeutic amount of sodium bicarbonate, the oral care composition adapted to be applied to the teeth and gums,
wherein the hydrodynamic power generated within the oral cavity by the tool in combination with the oral care composition removes biofilm from accessible surfaces and hard-to-reach surfaces of the teeth and gums, and
wherein the sodium bicarbonate in the oral care composition when combined with the hydrodynamic power more effectively removes biofilm.

10. The kit of claim 9, wherein the tool is a power toothbrush, an ultrasonic scaler, or a dental water irrigator.

11. The kit of claim 9, wherein the oral care composition is free of antimicrobial agents.

12. The kit of claim 9, wherein the oral care composition is a toothpaste and a concentration of sodium bicarbonate in the oral care composition is between 24% and 51% by weight.

13. The method of claim 9, wherein the oral care composition is a gel, mouthwash, or other liquid form and a concentration of sodium bicarbonate in the oral care composition is between 8% and 17% by weight.

14. The method of claim 9, wherein a concentration of sodium bicarbonate in the oral care composition is between 8.0% by weight and 51% by weight.

15. A combination for treating teeth and gums within an oral cavity, the combination comprising:
a tool that generates hydrodynamic power; and
an oral care composition including a therapeutic amount of sodium bicarbonate, the oral care composition adapted to be applied to the teeth and gums,
wherein the hydrodynamic power generated by the tool within an oral cavity in combination with the oral care composition removes biofilm from accessible surfaces and hard-to-reach surfaces of the teeth and gums, and
wherein the sodium bicarbonate in the oral care composition when combined with the hydrodynamic power more effectively removes biofilm.

16. The combination of claim 15, wherein the tool is a power toothbrush including a handle, a plurality of bristles supported by the handle, and a power source supported by the handle, the power source being configured to drive the movement of the plurality of bristles, the plurality of bristles being configured to contact surfaces of the teeth to remove biofilm therefrom and generate hydrodynamic power that removes biofilm from hard-to-reach surfaces of the teeth and gums.

17. The combination of claim 16, wherein the power toothbrush is a sonic toothbrush.

18. The combination of claim 15, wherein the oral care composition is free of antimicrobial agents.

19. The combination of claim 15, wherein the oral care composition is a toothpaste and a concentration of sodium bicarbonate in the oral care composition is between 24% and 51% by weight.

20. The combination of claim 15, wherein a concentration of sodium bicarbonate in the oral care composition is between 8.0% by weight and 51% by weight.
